(19) United States
(12) Patent Application Publication
(10) Pub. No.: US 2004/0142416 A1
(43) Pub. Date: Jul. 22, 2004

(54) Treatment for phenylketonuria

(76) Inventors: Philip J. Laipis, Gainesville, FL (US); Terence R. Flotte, Gainesville, FL (US)

Correspondence Address: Stanley A. Kim, Ph.D., Esq., Akerman Senterfitt, Suite 400, 222 Lakeview Avenue, West Palm Beach, FL, 33402-3188, US

(21) Appl. No.: 10/427,181

(22) Filed: Apr. 30, 2003

Related U.S. Application Data
(60) Provisional application No. 60/376,598, filed on Apr. 30, 2002.

(51) Int. Cl. C07H 21/04; C12N 9/06; C12N 15/861

(52) U.S. Cl. 435/69.1; 435/456; 435/191; 435/320.1; 435/325; 536/23.2

(57) ABSTRACT

Phenylalanine hydroxylase deficiency in a subject is corrected by administering to the subject rAAV-based vectors that include a sequence encoding functional phenylalanine hydroxylase. Ribozymes are used to reduce expression of defective phenylalanine hydroxylase in a cell.

### CROSS-REFERENCE TO RELATED APPLICATIONS

The present application claims the priority of U.S. provisional patent application No. 60/376,598 filed on Apr. 30, 2002.

### STATEMENT AS TO FEDERALLY SPONSORED RESEARCH

This invention was made with U.S. government support under grant numbers DK58327, GM50032, and AI045875 all awarded by the National Institutes of Health. The U.S. government may have certain rights in the invention.

### FIELD OF THE INVENTION

The invention relates generally to the fields of molecular biology, gene therapy, and medicine. More particularly, the invention relates to a gene therapy-based treatment for phenylketonuria (PKU).

### BACKGROUND OF THE INVENTION

PKU is one of the most common genetic disorders of man. This metabolic disease was initially characterized in two mentally retarded siblings by the Norwegian Asbjörn Fölling in 1934, but the defect was not identified biochemically until 1947 (Jervis, G. A., J. Biol. Chem. 169:651-656, 1947). PKU is usually caused by a single-gene defect in the enzyme phenylalanine hydroxylase (PAH), which results in elevated serum Phe levels. PAH converts Phe to tyrosine in vertebrates. In the absence of PAH, the only other mechanisms to remove Phe are protein synthesis and a minor degradative path involving the deamination and oxidative decarboxylation of the alanine side chain, which yields the characteristic phenyllactate and phenylacetate seen in urine of PKU patients. Unfortunately, a typical diet contains more Phe than can be eliminated in the absence of PAH. The resulting accumulation of Phe in PKU patients leads to a number of symptoms including abnormal brain development and severe mental retardation. (Kaufman, Proc Nat'l Acad Sci USA 96: 3160-3164, 1999).

Conventional treatments for PKU involve dietary restriction of phenylalanine (Phe) (Woolf et al. Br. J. Med. 1:57-64, 1957). While partially successful, such dietary restriction is difficult to maintain and does not correct the underlying defect. Despite the tremendous advances in understanding the biochemistry, molecular biology, and genetics of PKU, little progress has been made in developing new treatments for the disorder.

### SUMMARY

The invention relates to the development of a method for treating an animal suffering from PKU. In the method, recombinant adenovirus-associated virus (rAAV) vectors carrying PAH genes are delivered to the liver of an animal with PKU. In a mouse model of PKU, this method resulted in stable reduction of serum Phe levels to near-normal levels in mice. In addition, expression of defective PAH that can prevent the normal function of wild-type PAH was reduced by delivering anti-PAH ribozymes using rAAV.

Accordingly, the invention features a nucleic acid that includes a phenylalanine hydroxylase-modulating sequence interposed between a first AAV inverted terminal repeat and second AAV inverted terminal repeat. The phenylalanine hydroxylase-modulating sequence can be (a) a polynucleotide encoding a phenylalanine hydroxylase protein and/or (b) a catalytic polynucleotide that reduces expression of a phenylalanine hydroxylase protein (e.g., a ribozyme). The nucleic acid can be a vector such as a plasmid vector. It can also be contained within an rAAV virion.

The phenylalanine hydroxylase protein encoded by the polynucleotide can be capable of catalyzing the intracellular conversion of phenylalanine to tyrosine. For example, it can be a wild-type mammalian (e.g., human) phenylalanine hydroxylase protein.

The nucleic acid of the invention can include various regulatory elements such as a promoter operably lined to the phenylalanine hydroxylase-modulating sequence; an enhancer element; an intron; or a woodchuck hepatitis virus post-transcriptional element.

The invention also features a cell into which the nucleic acid of the invention has been introduced. The cell can be a mammalian cell such as a mammalian a liver cell.

In another aspect, the invention features a method for modulating phenylalanine hydroxylase activity in a cell. This method includes the step of administering to the cell an effective amount of the nucleic acid of the invention. For example, this method can include introducing into the cell both (a) the polynucleotide encoding a phenylalanine hydroxylase protein and (b) the catalytic polynucleotide that reduces expression of a phenylalanine hydroxylase protein. In the method, the cell can be located in an in vitro culture or within an animal subject (e.g., one having a defect in a phenylalanine hydroxylase gene).

Unless otherwise defined, all technical terms used herein have the same meaning as commonly understood by one of ordinary skill in the art to which this invention belongs.

By the term “gene” is meant a nucleic acid molecule that codes for a particular protein, or in certain cases a functional or structural RNA molecule.

As used herein, a “nucleic acid:, “nucleic acid molecule”, or “polynucleotide” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules.

As used herein, “protein” or “polypeptide” are used synonymously to mean any peptide-linked chain of amino acids, regardless of length or post-translational modification, e.g., glycosylation or phosphorylation.

When referring to a nucleic acid molecule or polypeptide, the term “native” refers to a naturally-occurring (e.g., a wild-type; “WT”) nucleic acid or polypeptide.

As used herein, the term “vector” refers to a nucleic acid molecule capable of transporting another nucleic acid to which it has been linked. Vectors capable of directing the expression of genes to which they are operatively linked are referred to herein as “expression vectors.”

A first nucleic-acid sequence is “operably” linked with a second nucleic-acid sequence when the first nucleic-acid sequence is placed in a functional relationship with the second nucleic-acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

As used herein, the phrase “expression control sequence” refers to a nucleic acid that regulates the replication, transcription and translation of a coding sequence in a recipient cell. Examples of expression control sequences include promoter sequences, polyadenylation (pA) signals, introns, transcription termination sequences, enhancers, upstream regulatory domains, origins of replication, and internal ribosome entry sites (“IRES”). The term “promoter” is used herein to refer to a DNA regulatory sequence to which RNA polymerase binds, initiating transcription of a downstream (3′ direction) coding sequence.

By the term “pseudotyped” is meant a nucleic acid or genome derived from a first AAV serotype that is encapsidated or packaged by an AAV capsid containing at least one AAV Cap protein of a second serotype. By “AAV inverted terminal repeats”, “AAV terminal repeats, “ITRs”, and “TRs” are meant those sequences required in cis for replication and packaging of the AAV virion including any fragments or derivatives of an ITR which retain activity of a full-length or WT ITR.

By the terms “AAV inverted terminal repeats”, “AAV terminal repeats, “ITRs”, and “TRs” are meant those sequences required in cis for replication and packaging of the AAV virion. The terms include any fragments or derivatives of a ITR which retain activity of a WT (e.g., full-length) ITR.

As used herein, the terms “rAAV vector” and “recombinant AAV vector” refer to a recombinant nucleic acid derived from an AAV serotype, including without limitation, AAV1, AAV2, AAV3, AAV4, AAV5, AAV6, AAV7, etc. rAAV vectors can have one or more of the AAV WT genes deleted in whole or in part, preferably the rep and/or cap genes, but retain functional flanking ITR sequences.

A “recombinant AAV virion,” or “rAAV virion” is defined herein as an infectious, replication-defective virus composed of an AAV protein shell encapsulating a heterologous nucleotide sequence flanked on both sides by AAV ITRs.

When referring to a nucleic acid, the term “heterologous” refers to a first nucleic acid sequence joined to a second nucleotide sequence in an order that does not generally occur naturally.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a highly schematic illustration of two PAH rAAV vectors useful in the invention.

FIG. 2 is a graph measuring PAH enzyme activity present in mouse liver homogenate, untransfected 293 cells, or 293 cells transfected with human or mouse CB-PAH cDNA constructs. Dashed lines: no Phe in assay; solid lines: Phe present.

FIG. 3 is a series of graphs of Phe levels mice treated with various rAAV vectors. (A) male Pah^{enu2} −/− mice treated with an rAAV vector containing the mouse PAH gene. (B) female Pah^{enu2} −/− mice treated with an rAAV vector containing the mouse PAH gene. (C) Male Pah^{enu2} −/− mice treated with a rAAV vector containing the human PAH gene.

FIG. 4 is a graph of Phe levels in male Pah^{enu2} mice receiving successful PKU gene therapy. Vector dose is shown as I.U.×10⁻¹⁰.

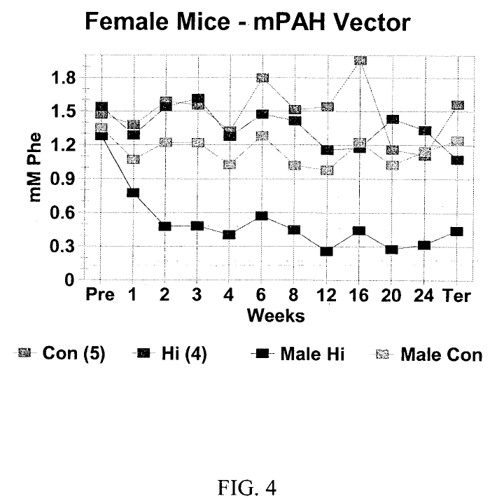

FIG. 5 is a schematic illustration showing a ribozyme contacting a mouse PAH RNA target.

FIG. 6 is a highly schematic illustration of an anti-PAH ribozyme expression vector.

FIG. 7 is a graph showing the results from an in vitro experiment in which the rAAV ribozyme vector was introduced into cells with the rAAV-PAH described herein. The Y axis represents PAH enzymatic activity.

### DETAILED DESCRIPTION

The invention provides compositions and methods relating to rAAV-mediated gene transfer of genetic material including a phenylalanine hydroxylase-modulating sequence into a host cell or organism lacking normal PAH activity. The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Nucleic Acids For Modulating PAH Expression

rAAV-mediated transfer of an agent that modulates PAH expression in a cell or animal is accomplished using a nucleic acid that includes a polynucleotide encoding the agent interposed between two AAV ITRs. The PAH-modulating agent can be a functional PAH protein (i.e., one that catalyzes the conversion of Phe to tyrosine) or a catalytic polynucleotide that reduces expression of non-functional PAH (e.g., a ribozyme, an antisense molecule, or a small interfering RNA).

The PAH-encoding polynucleotide sequence can take many different forms. For example, the sequence may be a native mammalian PAH nucleotide sequence such as the mouse or human PAH-encoding sequences deposited with Genbank as accession numbers X59142 and U49897. The PAH-encoding nucleotide sequence may also be a non-native coding sequence which, as a result of the redundancy or degeneracy of the genetic code, encodes the same polypeptide as does a native mammalian PAH nucleotide sequence. Other PAH-encoding nucleotide sequences within the invention are those that encode fragments, analogs, and derivatives of a native PAH protein. Such variants may be, e.g., a naturally occurring allelic variant of a native PAH-encoding nucleic acid, a homolog of a native PAH-encoding nucleic acid, or a non-naturally occurring variant of native PAH-encoding nucleic acid. These variants have a nucleotide sequence that differs from native PAH-encoding nucleic acid in one or more bases. For example, the nucleotide sequence of such variants can feature a deletion, addition, or substitution of one or more nucleotides of a native PAH encoding nucleic acid. Nucleic acid insertions are preferably of about 1 to 10 contiguous nucleotides, and deletions are preferably of about 1 to 30 contiguous nucleotides. In most applications of the invention, the polynucleotide encoding a PAH substantially maintains the ability to convert phenylalanine to tyrosine.

The PAH-encoding nucleotide sequence can also be one that encodes a PAH fusion protein. Such a sequence can be made by ligating a first polynucleotide encoding a PAH protein fused in frame with a second polynucleotide encoding another protein (e.g., one that encodes a detectable label). Polynucleotides that encode such fusion proteins are useful for visualizing expression of the polynucleotide in a cell.

The catalytic polynucleotide that reduces expression of non-functional PAH (e.g., a ribozyme, an antisense molecule, or a small interfering RNA) can also take many different forms. These are described in more detail below.

In order to facilitate its long term expression, the polynucleotide encoding the agent that modulates PAH activity is interposed between first and second AAV ITRs. AAV ITRs are found at both ends of a WT AAV genome, and serve as the origin and primer of DNA replication. ITRs are required in cis for AAV DNA replication as well as for rescue, or excision, from prokaryotic plasmids. The AAV ITR sequences that are contained within the nucleic acid can be derived from any AAV serotype (e.g., 1, 2, 3, 4, 5, 6 and 7) or can be derived from more than one serotype. For use in a vector, the first and second ITRs should include at least the minimum portions of a WT or engineered ITR that are necessary for packaging and replication.

In addition to the AAV ITRs and the polynucleotide encoding the agent that modulates PAH activity, the nucleic acids of the invention can also include one or more expression control sequences operatively linked to the polynucleotide encoding the agent that modulates PAH activity. Numerous such sequences are known. Those to be included in the nucleic acids of the invention can be selected based on their known function in other applications. Examples of expression control sequences include promoters, insulators, silencers, response elements, introns, enhancers, initiation sites, termination signals, and pA tails.

To achieve appropriate levels of the agent that modulates PAH activity, any of a number of promoters suitable for use in the selected host cell may be employed. For example, constitutive promoters of different strengths can be used. Expression vectors and plasmids in accordance with the present invention may include one or more constitutive promoters, such as viral promoters or promoters from mammalian genes that are generally active in promoting transcription. Examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Examples of constitutive mammalian promoters include various housekeeping gene promoters, as exemplified by the β-actin promoter. As described in the examples below, the chicken beta-actin (CB) promoter has proven to be a particularly useful constitutive promoter for expressing PAH.

Inducible promoters and/or regulatory elements may also be contemplated for use with the nucleic acids of the invention. Examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are useful in certain embodiments of the invention. Examples of such promoters that may be used with the expression vectors of the invention include promoters such as the PAH promoter itself, the albumin, alpha-I antitrypsin, or clotting factor IX promoters, which are specific for liver cells. Since PAH is also expressed in kidney, the PAH promoter, or kidney specific promoters such as elements of the nephrin (J Am Soc Nephrol, 14:352-8, 2003) or 25-OH-D(3) alpha-hydroxylase (J Cell Biochem 88:245-51, 2003) promoters may be useful in targeting PAH gene expression to this tissue.

### rAAV Vectors And Virions

The nucleic acids of the invention may be incorporated into vectors and/or virions in order to facilitate their introduction into a cell. rAAV vectors useful in the invention are recombinant nucleic acid constructs that include (1) a heterologous sequence to be expressed (e.g., a polynucleotide encoding an agent that modulates PAH activity) and (2) viral sequences that facilitate integration and expression of the heterologous genes. The viral sequences may include those sequences of AAV that are required in cis for replication and packaging (e.g., functional ITRs) of the DNA into a virion. In preferred applications, the heterologous gene encodes PAH (e.g., Pah), which is useful for correcting a PAH-deficiency in a cell. Such rAAV vectors may also contain marker or reporter genes. Useful rAAV vectors have one or more of the AAV WT genes deleted in whole or in part, but retain functional flanking ITR sequences. The AAV ITRs may be of any serotype (e.g., derived from serotype 2) suitable for a particular application. Methods for using rAAV vectors are discussed, for example, in Tal, J., J. Biomed. Sci. 7:279-291, 2000 and Monahan and Samulski, Gene delivery 7:24-30, 2000.

The nucleic acids and vectors of the invention may be incorporated into an rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described. See, e.g., U.S. Pat. Nos. 5,173,414, 5,139,941, 5,863,541, and 5,869,305, 6,057,152, 6,376,237; Rabinowitz et al., J. Virol. 76:791-801, 2002; and Bowles et al., J. Virol. 77:423-432, 2003.

rAAV vectors and virions useful in the invention include those derived from a number of AAV serotypes. The rAAV vectors used in the invention may be derived from any of several AAV serotypes including 1, 2, 3, 4, 5, 6, and 7. Because of wide construct availability and extensive characterization, serotype 2 rAAV vectors are preferred from many applications. For targeting liver, serotype 5 rAAV vectors are preferred as these has shown enhanced infectivity of hepatocytes. Construction and use of AAV vectors and AAV proteins of different serotypes are discussed in Chao et al., Mol. Ther. 2:619-623, 2000; Davidson et al., Proc Nat'l Acad Sci USA 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

The rAAV virions described above may be administered to a cell (e.g., cell within a subject) more than once. For repeat administration, the use of rAAV2 vectors pseudotyped (i.e., encapsulated) with capsid proteins from non-AAV2 serotypes (e.g., 1, 3, 4, 5, 6, 7) may be particularly useful for mitigating an immune response against the vector. Pseudotyped vectors of the invention include AAV2 vectors pseudotyped with a capsid gene derived a serotype other than 2 (e.g., AAV1, AAV3, AAV4, AAV5, AAV6 or AAV7 capsids). For example, preferred pseudotyped vectors of the invention are AAV2 vectors encoding PAH pseudotyped with a capsid gene derived from AAV serotype 5, as serotype 5 has shown enhanced infectivity of hepatocytes compared to other serotypes. Techniques involving the construction and use of pseudotyped rAAV virions are known in the art and are described in Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet. 10:3075-3081, 2001.

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45, 2000. Other rAAV virions that can be used in methods of the invention include those capsid hybrids that are generated by molecular breeding of viruses as well as by exon shuffling. See Soong et al., Nat. Genet. 25:436-439, 2000; and Kolman and Stemmer Nat. Biotechnol. 19:423-428, 2001.

### Antisense, Ribozyme, Triplex, and RNA Interference Techniques

Another aspect of the invention relates to the use of a catalytic polynucleotide to inhibit expression of endogenous, defective PAH (that might compete with functional PAH) in a cell. Examples of such catalytic polynucleotide include antisense molecules, ribozymes, or interfering RNAs.

Antisense nucleic acid molecules within the invention are those that specifically hybridize (e.g., bind) under cellular conditions to cellular mRNA and/or genomic DNA encoding a PAH protein (e.g., a mutant PAH displaying less than wild-type ability to covert Phe to tyrosine) in a manner that inhibits expression of the PAH protein, e.g., by inhibiting transcription and/or translation. The binding may be by conventional base pair complementarity, or, for example, in the case of binding to DNA duplexes, through specific interactions in the major groove of the double helix.

Antisense constructs can be delivered, for example, as an expression vector (e.g., one of the rAAV vectors described above) which, when transcribed in the cell, produces RNA which is complementary to at least a unique portion of the cellular mRNA which encodes a PAH protein. Alternatively, the antisense construct can take the form of an oligonucleotide probe generated ex vivo which, when introduced into a PAH-expressing cell, causes inhibition of PAH expression by hybridizing with an mRNA and/or genomic sequences coding for PAH. Such oligonucleotide probes are preferably modified oligonucleotides that are resistant to endogenous nucleases, e.g. exonucleases and/or endonucleases, and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668. With respect to antisense DNA, oligodeoxyribonucleotides derived from the translation initiation site, e.g., between the −10 and +10 regions of an PAH encoding nucleotide sequence, are preferred.

Antisense approaches involve the design of oligonucleotides (either DNA or RNA) that are complementary to Pah mRNA. The antisense oligonucleotides will bind to Pah mRNA transcripts and prevent translation. Absolute complementarity, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. (1994) Nature 372:333). Therefore, oligonucleotides complementary to either the 5′ or 3′ untranslated, non-coding regions of a Pah gene could be used in an antisense approach to inhibit translation of endogenous Pah mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′, 3′ or coding region of Pah mRNA, antisense nucleic acids should be at least six nucleotides in length, and are preferably less than about 100 and more preferably less than about 50, 25, 17 or 10 nucleotides in length.

Ribozyme molecules designed to catalytically cleave Pah mRNA transcripts can also be used to prevent translation of Pah mRNA and expression of PAH (See, e.g., PCT Publication No. WO 90/11364, published Oct. 4, 1990; Sarver et al., Science 247:1222-1225, 1990 and U.S. Pat. No. 5,093,246). While ribozymes that cleave mRNA at site-specific recognition sequences can be used to destroy Pah mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, (Nature 334:585-591, 1988). Preferably the ribozyme is engineered so that the cleavage recognition site is located near the 5′ end of Pah mRNA; i.e., to increase efficiency and minimize the intracellular accumulation of non-functional mRNA transcripts.

Ribozymes within the invention can be delivered to a cell using a vector (e.g., rAAV) as described above. In preferred methods of the invention, a ribozyme produced by a rAAV vector is introduced into PKU subjects along with the rAAV vector producing functional PAH protein. The rAAV_PAH sequence can be “hardened” to make it resistant to ribozyme destruction. For a review of this topic, see Mautino, M. R. Curr. Gene Ther. 2:23-43, 2002. Thus, the mRNA encoding the non-functional PAH will be degraded, while the introduced functional PAH mRNA will not. This topic is reviewed in Mautino M R., Curr Gene Ther. 2:23-43, 2002.

Endogenous Pah gene expression can also be reduced by inactivating or “knocking out” the Pah gene or its promoter using targeted homologous recombination. See, e.g, Kempin et al., Nature 389: 802, 1997; Smithies et al., Nature 317:230-234, 1985; Thomas and Capecchi, Cell 51:503-512, 1987; and Thompson et al., Cell 5:313-321, 1989. For example, a mutant, non-functional Pah variant (or a completely unrelated DNA sequence) flanked by DNA homologous to the endogenous Pah gene (either the coding regions or regulatory regions of the PAH gene) can be used, with or without a selectable marker and/or a negative selectable marker, to transfect cells that express PAH in vivo.

Alternatively, endogenous Pah gene expression might be reduced by targeting deoxyribonucleotide sequences complementary to the regulatory region of the Pah gene (i.e., the Pah promoter and/or enhancers) to form triple helical structures that prevent transcription of the Pah gene in target cells. (See generally, Helene, C. Anticancer Drug Des. 6(6):569-84, 1991; Helene, C., et al., Ann. N.Y. Acad. Sci. 660:27-36, 1992; and Maher, L. J. Bioassays 14(12):807-15, 1992).

Another technique that may be employed to modulate PAH expression is RNA interference (RNAi). Chuang and Meyerowicz, Proc. Nat'l Acad. Sci. USA, 97:4985, 2000. In this technique, double-stranded RNA (dsRNA)-expressing constructs are introduced into a cell using a suitable method of transformation. In this manner, such dsRNA is persistent and inherited. By selecting appropriate sequences (e.g., those corresponding to Pah), expression of dsRNA can interfere with accumulation of endogenous mRNA encoding a target protein (e.g., PAH).

### Modulating PAH Levels In A Cell

The nucleic acids, vectors, and virions described above can be used to modulate levels of PAH in a cell. The method includes the step of administering to the cell a composition including a nucleic acid that includes a polynucleotide encoding an agent that modulates PAH expression interposed between two AAV ITRs. The cell can be from any animal into which a nucleic acid of the invention can be administered. Mammalian cells (e.g., human beings, dogs, cats, pigs, sheep, mice, rats, rabbits, cattle, goats, etc.) from a subject with PKU are typical target cells for use in the invention.

### Increasing PAH Activity In A Subject

The nucleic acids, vectors, and virions described above can be used to modulate levels of functional PAH in an animal subject. The method includes the step of providing an animal subject and administering to the animal subject a composition including a nucleic acid that includes a polynucleotide encoding an agent that modulates PAH expression interposed between two AAV ITRs. The subject can be any animal into which a nucleic acid of the invention can be administered. For example, mammals (e.g., human beings, dogs, cats, pigs, sheep, mice, rats, rabbits, cattle, goats, etc.) are suitable subjects. The methods and compositions of the invention are particularly applicable to PKU animal subjects.

The compositions described above may be administered to animals including human beings in any suitable formulation by any suitable method. For example, rAAV virions (i.e., particles) may be directly introduced into an animal, including by intravenous injection, intraperitoneal injection, or in situ injection into target tissue. For example, a conventional syringe and needle can be used to inject an rAAV virion suspension into an animal. Depending on the desired route of administration, injection can be in situ (i.e., to a particular tissue or location on a tissue), intramuscular, intravenous, intraperitoneal, or by another parenteral route. Parenteral administration of virions by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the rAAV virions may be in powder form (e.g., lyophilized) for constitution with a suitable vehicle, for example, sterile pyrogen-free water, before use.

To facilitate delivery of the rAAV virions to an animal, the virions of the invention can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly preferred for delivery of virions to human subjects. Methods for making such formulations are well known and can be found in, for example, Remington's Pharmaceutical Sciences.

In addition to the formulations described previously, the virions can also be formulated as a depot preparation. Such long acting formulations may be administered by implantation (for example subcutaneously or intramuscularly) or by IM injection. Thus, for example, the virions may be formulated with suitable polymeric or hydrophobic materials (for example as an emulsion in an acceptable oil) or ion exchange resins, or as sparingly soluble derivatives.

Similarly, rAAV vectors may be administered to an animal subject using a variety of methods. rAAV vectors may be directly introduced into an animal by peritoneal administration (e.g., intraperitoneal injection, oral administration), as well as parenteral administration (e.g., intravenous injection, intramuscular injection, and in situ injection into target tissue). Methods and formulations for parenteral administration described above for rAAV virions may be used to administer rAAV vectors.

Ex vivo delivery of cells transduced with rAAV virions is also provided for within the invention. Ex vivo gene delivery may be used to transplant rAAV-transduced host cells back into the host. A suitable ex vivo protocol may include several steps. A segment of target tissue (e.g., muscle tissue) may be harvested from the host and rAAV virions may be used to transduce a PAH-encoding nucleic acid into the host's cells. These genetically modified cells may then be transplanted back into the host. Several approaches may be used for the reintroduction of cells into the host, including intravenous injection, intraperitoneal injection, or in situ injection into target tissue. Microencapsulation of cells transduced or infected with rAAV modified ex vivo is another technique that may be used within the invention. Autologous and allogeneic cell transplantation may be used according to the invention.

### Effective Doses

The compositions described above are preferably administered to a mammal in an effective amount, that is, an amount capable of producing a desirable result in a treated subject (e.g., increasing wild-type PAH activity in the subject). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one animal depends on many factors, including the subject's size, body surface area, age, the particular composition to be administered, time and route of administration, general health, and other drugs being administered concurrently. The dosage for any one animal will also depend on the particular PKU mutation found in the animal. It is expected that an appropriate dosage for intravenous administration of the compositions would be in the range of about 1×10¹⁰ to 1×10¹⁶ vector genomes/kg. For a 70 kg human, approximately 1.5×10¹⁵ vector genomes in a volume in the range of about 1-100 ml (e.g., 5-50 ml) delivered via catheter into the hepatic artery is presently believed to be an appropriate dose.

### EXAMPLES

The present invention is further illustrated by the following specific examples, which should not be construed as limiting the scope or content of the invention in any way.

### Example 1

### Cloning the Human and Mouse PAH Genes

PCR amplification methods were used to isolate seven independent human cDNA clones from a human fetal liver cDNA library (Clontech HL1151x). All clones were subcloned into a rAAV expression construct (see below) and transfected into 293 cells (human fetal kidney fibroblasts) for assessment of enzyme activity. Three clones expressed biologically active PAH; they were sequenced and compared to the published human PAH sequence (Genbank accession U49897, Konecki et al., Biochemistry 31:8363-8368, 1992; Nowacki et al., Nuc. Acids Res. 25:139-142, 1997). All have four silent transitions presumably resulting from human population polymorphism. Similarly, five mouse cDNA clones were isolated by PCR directly from cDNA (Clontech, adult Balb/c liver). The clones were sequenced and compared to the published mouse PAH sequence (Genbank accession X59142, Ledley et al., 1990). All have two silent transitions, and two coding changes, a GA->AG inversion (R383Q) and a A−>T transversion (N4321). Both changes make the mouse sequence identical to the human PAH sequence at those positions and may represent sequence errors in the published sequence. All had additional sequence changes, presumably caused by errors during PCR amplification or reverse transcription. An expression vector containing the correct sequence was generated by digestion and assembly of appropriate restriction enzyme fragments from two of the original isolates. This clone has somewhat greater activity than the PAH expressing human clones, consistent with previous reports (Kaufman, 1997).

### Example 2

### AAV Recombinant PAH Vector Construction and Expression Testing

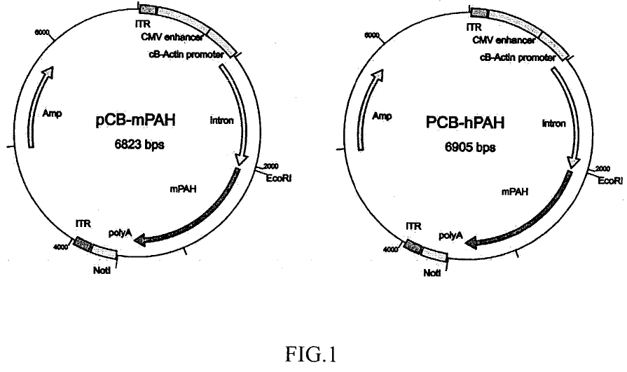

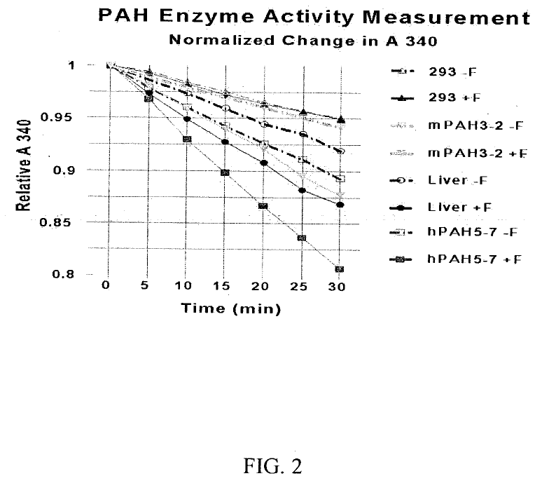

Both the human and mouse cDNA sequences were inserted into an AAV-based expression vector that allows packaging of a rAAV vector (Zolotukhin et al., Gene Therapy 6:973-985, 1999). Enzyme expression is driven by a hybrid CMV enhancer-chicken beta-actin (CB) promoter construct containing a hybrid chicken beta-actin/rabbit beta-globin intron, FIG. 1 (Niwa et al., Gene 108:13-199, 1991). The vector backbone is based on p43hAAT (Song et al. Gene Ther. 8:1299-1306, 2001); the human AAT coding sequence was removed and the PAH genes inserted. Choice of the CB promoter for PAH expression was determined by the efficiency of this promoter in previous studies (Song et al. Gene Ther. 8:1299-1306, 2001). Purified DNA from the constructs was used in a calcium phosphate-mediated transient transfection assay in 293 cells. After 48 hours, approximately 3×10⁶ transfected cells were harvested, homogenized, a clarified supernatant prepared, and enzyme activity measured spectrophotometrically. The enzyme assay is based on the phenylalanine-dependent linked reduction of NADH in the presence of excess 6-methyltetrahydropterin and dihydropteridine reductase (DHPR) (Kaufman and Fisher, J. Biol. Chem. 245:4745-4750, 1970; Parniak and Kaufman, J. Biol. Chem. 256:6876-6882, 1981; McDonald et al., Proc Nat'l Acad Sci USA 87:1965-1967, 1990). FIG. 2 shows a typical assay.

This assay was used to determine the specific activity of our rAAV-CB-PAH clones compared to the specific activity of WT, heterozygote, and Pah^{enu2} mouse liver (Table 1). Enzyme activity after infection of 293 cells with packaged rAAV vector is also shown. The value for endogenous mouse liver PAH activity measured here is very similar to that determined by McDonald and co-workers, 12.2 units/mg protein (McDonald et al., Proc Nat'l Acad Sci USA 87:1965-1967, 1990).

TABLE 1
Phenylalanine Hydroxylase Enzyme Assays

| Cell Source | # of Samples^{a} | Units/mg protein^{b} |
|---|---|---|
| Mouse Liver +/+^{c} | >30 | 6.8 ± 1.1 |
| Mouse Liver +/−^{c} | 10 | 1.8 ± 0.9 |
| Pah^{enu2} Mouse Liver^{c} | 6 | −0.0 ± 0.2 |
| CB hPAH 5-7^{d} | 5 | 16.4 ± 7.0 |
| CB mPAH 3-2^{d} | 5 | 17.0 ± 8.3 |
| UF5^{d} | 9 | 0.4 ± 0.9 |
| 293 (untransfected) | 5 | −0.1 ± 0.4 |
| CB hPAH M.O.I. 20^{e} | 2 | 4.2 |
| CB mPAH M.O.I. 20^{e} | 2 | 6.9 |

^{a}The number of samples for each construct or tissue represents a different experiment.
^{b}Units of enzyme activity are expressed as micromoles NADH oxidized per minute; mean and standard deviation are shown.
^{c}Mouse liver assays are carried out on frozen aliquots of cell extract. A cell extract is assayed once and discarded although enzyme activity is stable for at least 3-4 freeze-thaw cycles.
^{d}The CB hPAH and CD mPAH constructs were transfected into 293 cells using calcium phosphate. UF5 is a green fluorescent protein (GFP)-expressing AAV-based vector used as a transfection control; it contains no PAH coding elements.
^{e}The packaged recombinant vector was used to infect 293 cells at the indicated multiplicity of infection (M.O.I.), with co-infection with adenovirus type 5, M.O.I. 5.

Dilution assays showed that assay sensitivity allowed a clean detection of 10-20% transfected cells against a background of non-transfected 293 cells.

### Example 3

### Recombinant AAV Vector Packaging

Human and mouse PAH vector DNAs were used to make recombinant virions via a two-plasmid transfection process (Zolotukhin et al., Gene Therapy 6:973-985, 1999). Vector titers range from 2.0×10¹⁰ I.U./ml to 3.5×10¹¹ I.U./ml; particle to infectivity ratios range from 10-20. WT AAV was non-detected. All preparations showed enzyme activity in 293 cells at M.O.I.s of 20 (Table 1).

### Example 4

### The PKU Mouse Model

The Pah^{enu2} mouse was created by Shedlovsky and collaborators (1993) using ethylnitrourea to mutate male BTBR mice, who were then mated to normal BTBR females. Offspring were crossed to Pah^{enu1} mice (McDonald et al., Proc Nat'l Acad Sci USA 87:1965-1967, 1990) and the mutation identified by screening for high serum Phe levels. Pah^{enu1} mice had been created by a similar mutagenesis followed by two rounds of inbreeding to identify progeny that failed to clear a Phe load; they are a less severe phenotype at this locus. The Pah^{enu2} mouse lacks detectable PAH activity although an immunologically cross-reactive protein is detectable; levels of mRNA have been reported to be only 1% of normal (McDonald et al., Proc Nat'l Acad Sci USA 87:1965-1967, 1990). The mice have now been backcrossed for many generations. The mutation is a T to C transition changing Phe 263 to Ser, and incidentally creates a new Alw26I restriction site (McDonald and Charlton, Genomics 39:402-405, 1997). The mutation is in a conserved region of exon 7 and several known human PKU mutants have been identified at the same site. The mutation results in a phenotype that closely mimics the human disorder. Pah^{enu2} mice have elevated Phe levels, hypopigmentation, show behavior and movement abnormalities (elevated response to stress, ataxia), have a slower growth rate than +/− littermates, small heads and eyes, reduced fertility, and most importantly, females show a maternal PKU syndrome with pups exhibiting perinatal mortality and cardiovascular defects (McDonald et al., Pediat. Res. 42:103-107, 1997). Interestingly, one finding not seen in Pah^{enu2} mice is the gross structural abnormalities in brain structure and demyclination reported in adult human patients (Kornguth et al., Neuroimage 1:220-229, 1994). However, the study described herein was carried out with 7 week old Pah^{enu2} mice. In the human studies cited by Kornguth and collaborators (Kornguth et al., Neuroimage 1:220-229, 1994), all of the human patients were older than 13 and many over 20; studies cited by Scriver et al. “The Hyperphenylalaninemias,” In The Metabolic and Molecular Basis of Inherited Disease 7th edition, chap. 27:1015-1075, 1995) also used older patients. While 7 week old mice are pubertal, this may not closely correlate with a 20 year old human; a repetition of this study with older Pah^{enu2} mice is required. Even though gross defects were not visible, subtle changes in neuroanatomy, myelin turnover, and cell populations in the brain have been reported in Pah^{enu2} mice (Hommes, J. Inher. Metab. Dis. 16:962-974, 1993; and Dyer et al., J. Neuropathol. Exp. Neurol. 55:795-814, 1996). Recently, cognitive defects in odor discrimination reversal and latent learning have been demonstrated in Pah^{enu2} mice (Zagreda et al., J. Neurosci. 19:6175-6182, 1999). The Pah^{enu2} mouse is considered an excellent animal model of human PKU.

A breeding colony of Pah^{enu2} mice was established. Breeding uses both +/− and −/− males with +/−females; +/−×+/− breeding necessitates genotyping of normal-coated offspring. The Pah^{enu2} mutation is a F263S change in exon 7; creating a Alw26I restriction site (McDonald and Charlton, Genomics 39:402-405, 1997). To genotype offspring, an exon 7 genomic fragment was amplified and digested with Alw26I to confirm the Pah^{enu2} mutation.

In addition to the Pah^{enu2} genotype assay, a serum Phe assay was developed. A human Phe assay was modified to use 5 microliters of mouse serum, using Sigma assay kits and a Sequoia-Turner Model 450 Fluorometer, for day-to-day use. A microtiter plate assay using 7.5 microliters of serum to do triplicate assays was also developed. This assay was used for final analysis of experimental results. 50-75 microliters of blood can be obtained from the Pah^{enu2} mice weekly, without mortality; this is sufficient for the assays. Table 2 shows serum Phe levels in Pah^{enu2} mice from the colony as well as selected controls. Note that females are more severely affected than males.

TABLE 2
Serum Phenylalanine Assays

| Serum Source | # of Samples^{a} | mM Phe^{b} |
|---|---|---|
| Human +/+ | 15 (1) | 0.10 ± 0.02 |
| Human −/− | 11 (2) | 2.1 ± 0.30 |
| BTBR +/+ | 23 (8) | 0.10 ± 0.03 |
| Pah^{enu2} +/− | 15 (7) | 0.13 ± 0.03 |
| Pah^{enu2} −/− males | 14 (11) | 1.22 ± 0.16^{c} |
| Pah^{enu2} −/− females | 30 (13) | 1.72 ± 0.21^{c} |

^{a}Represents number of assays performed and, in parentheses, the number of individuals assayed.
^{b}Values represent the mean and standard deviation; The data for Pah^{enu2} −/− mice were obtained from 8 week or older adults; pups average 0.5 mM higher in Phe levels at weaning.
^{c}The difference between males and females is statistically significant (p 0.001).

### Example 5

### Portal Vein Injection of rAAV PAH Vectors

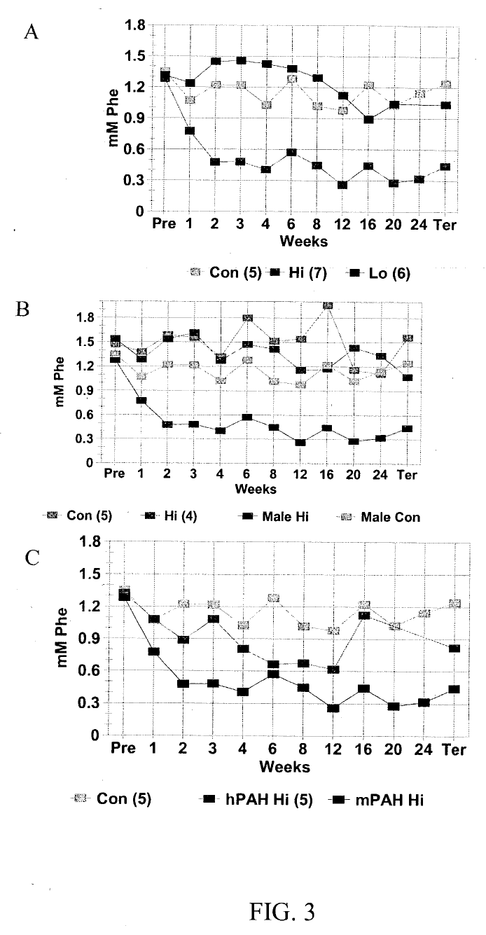

Efficient delivery of rAAV vectors to the liver is facilitated by injection into the portal vein. Over 60 such injections were carried out using both mouse and human PAH rAAV vectors at a range of doses, into both male and female Pah mice. Serum Phe levels in treated mice were then followed for up to 35 weeks, when the experiments were terminated for analysis. The results are presented in FIG. 3.

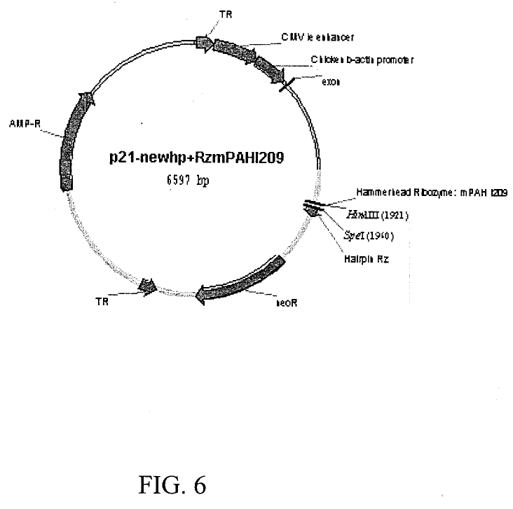

To summarize this data, seven male Pah^{enu2} mice treated with 7×10¹⁰ I.U. or higher doses of rAAV-CB-mPAH responded by lowering serum Phe levels into a therapeutic range. These mice also showed a reversal of the typical melanin biosynthetic defect seen in Pah^{enu2} mice, FIG. 6. In general, they appeared in better physical condition than non-responding males. FIG. 3 shows that at a vector dose of from 6×10¹⁰ to 1.5×10¹¹ I.U., all treated mice showed a lowering of serum Phe levels to a therapeutic range (below 0.6 mM) with several animals in the normal range (below 0.3 mM Phe). Melanin biosynthesis also resumed and coat color darkened. Vector doses of 3×10¹⁰ I.U. or below were ineffective.

Six male Pah^{enu2} mice treated with 3×10¹⁰ I.U. of rAAV-CB-mPAH did not respond. Four male Pah^{enu2} mice treated with 9-35×10¹⁰ I.U. of rAAV-CB-hPAH also did not respond, or, at best, showed a statistically non-significant drop in serum Phe levels, despite the approximately equal activity of the two vectors in an enzyme assay (Table 1, above). Given that the Pah^{enu2} mutation is a missense mutant and shows evidence of dominant-negative interactions, this non-response to a human protein in the context of mutant, mouse, non-functional subunits is understandable. More significant is the non-response of female Pah^{enu2} mice. Four female Pah^{enu2} mice received a 7×10¹⁰ I.U. or higher dose of rAAV-CB-mPAH vector; none showed a response to vector. Vector doses of up to 1.5×10¹¹ I.U. were ineffective in reducing serum Phe levels (FIG. 3B), in contrast to male Pah^{enu2} −/− mice. The gene therapy was technically successful since vector DNA is detectable in liver (by both quantitative PCR and in situ hybridization) at levels equivalent to those seen in male mice. All mice in these experiments were necropsied and examined histopathologically. There were no signs of vector-related pathology in any mice, consistent with previous data from rAAV gene therapy trials (Donsante et al. Gene Ther. 8:1343-6, 2001). FIG. 3C shows Phe levels in male Pah^{enu2} −/− mice treated with a rAAV vector containing the human PAH gene driven by the CB hybrid promoter. At vector doses of from 1.5×10¹¹ to 3.6×10¹¹ I.U., two mice showed a transient lowering of serum Phe levels to the therapeutic range (0.6 mM). Vector doses of 7×10¹⁰ I.U. or below were ineffective. Genotyping on tail DNA isolated from AA196769 showed expression persisted 10 weeks after rAAV treatment

Vector copy number in relation to delivered dose was determined by real-time “Taqman” PCR methods. There is a clear correlation between vector dose, response to therapy and vector/cell genome ratio in treated mice. Male mice who contained at least 1 vector copy per genome equivalent in liver showed a therapeutic response; to attain this level of vector content required a minimum of 7×10¹⁰ I.U. vector delivered via the portal vein. There were no differences in vector content per genome equivalent between males or females receiving the mouse PAH vector, nor males receiving the human PAH vector.

Distribution of the vector within the liver was examined by in situ hybridization. Two male mice who received vector doses of 1.5×10¹¹ I.U. showed about 25% (21-27%) of hepatocytes with a positive signal at 24 weeks post gene therapy. A vector saturation experiment similar to those of Miao et al., J. Virol. 74:3793-3803, 2000) suggested that a vector/genome ratio of 20 in total liver hepatocytes by quantitative PCR resulted in greater than 80% of hepatocytes showing a positive in situ hybridization signal. This result suggests that the in situ detection level was on the order of 5 vector genomes per hepatocyte.

### Example 6

### Female Pah^{enu2} Mice Did Not Respond to Gene Therapy

In the Pah^{enu2} mouse model, females did not respond to vector doses that were clearly therapeutic in males. To examining this disparity, the changes in serum Phe levels in female Pah^{enu2} mice subjected to gene therapy and hormonal modulation were examined. A cohort of female Pah^{enu2} mice were bled four times over a 5-6 week interval. They were then ovariectomized and further bled four times over the next 6-8 weeks. They then received 1.5×10¹¹ I.U. of rAAV-CBmPAH vector, via portal vein delivery, along with a subcutaneous time-release pellet containing either placebo, 17-beta estradiol or 5-alpha dihydrotestosterone (Innovative Research of America, Sarasota, Fla.).

The results showed that ovariectomy alone had no significant effect on serum Phe levels, suggesting the female sex hormones estrogen and progesterone had no major influence on Phe levels. Introduction of a rAAV-CBmPAH vector also had no effect in ovariectomized females or females given an estrogen implant. However, a female receiving rAAV vector plus a testosterone implant rapidly reduced her serum Phe levels into the therapeutic range, gained body mass, and reversed the defect in melanin biosynthesis.

### Example 7

### Enhancement of rAAV-mPAH Vector Activity by Modification of the Vector Sequence to Include a Woodchuck Hepatitis Virus Post-Transcriptional Response Element (WPRE)

A WPRE was added to the rAAV-mPAH vector sequence in the 3′ untranslated region of the PAH mRNA. In in vitro experiments in which vector was transfected into human 293 cells, this element increased vector production of PAH enzyme activity about 2-fold. This vector was also introduced into the Pah^{enu2} mouse model of PKU. In vivo, it was at least twice as efficient as the previous vector lacking this element (see Table 1). It was effective in lowering serum Phe levels to normal (below 0.12 mM) in three male mice, at a vector dose 40% of what was required with the initial construct. More importantly, this vector showed effectiveness in female mice, reducing serum Phe levels to a therapeutic level (˜0.6 mM Phe) at a vector dose of 8.7×10¹⁰ I.U., where the previous vector, at a dose of 1.5×10¹¹ showed no effect.

TABLE 3
Serum Phe levels in Pah^{enu2} mice
treated with rAAV-mPAH-WPRE vector

| sex | vector dose | prebleed | 2 weeks | 3 weeks |
|---|---|---|---|---|
| Male | 2.7 × 10¹⁰ I.U. | 1.14 mM | 0.14 mM | 0.08 mM |
| Female | 8.7 × 10¹⁰ I.U. | 1.15 mM | 0.98 mM | 0.61 mM |

PKU is defined as 1.0 mM or above; serum Phe levels in normal individuals range from 0.50 to 1.2 mM. A therapeutic range in PKU patients is generally regarded as below 0.4 mM, although in many individuals, 0.6 mM is acceptable.

### Example 8

### Carrier and PKU Mice Show Reduced Levels of PAH Protein

Evidence for dominant negative interference in PKU patients and in the Pah^{enu2} mouse model of PKU was presented in Example 2, in the form of enzyme activity measurements in WT (+/+), carrier (+/−) and PKU mice (−/−) (Table 4, PAH enzyme activity in normal, carrier, and PKU mice). It was confirmed that although the carrier and PKU mice have greatly reduced or no PAH activity, respectively, they also show reduced levels of PAH protein by Western blot analysis (reduced PAH protein levels in carrier and PKU mice). This data shows that the PKU mutation in these mice not only reduces activity in the carrier animal, with one normal and one mutant gene, but it reduces the amount of protein as well. Pah^{enu2} mice have detectable amounts of the non-functional F263S mutant protein, albeit at reduced levels. Since PAH is a tetrameric enzyme with four subunits, mixtures of normal and mutant subunits reduce both the stability and activity of the PAH protein. Thus, vector treatment of the PKU mutant animal required high levels of vector to overcome the effect of the two mutant genes (one paternal, one maternal) in the PKU mouse. This requirement was partially lowered by the use of the WPRE element described in Example 7.

TABLE 4
Reduced levels of PAH enzyme activity in liver extracts of
Pah^{enu2} mice compared to BTBT +/+ mice.

| Enzyme Source | Number of Samples | Units/mg Protein |
|---|---|---|
| Mouse liver +/+ (WT) | >30 | 8.8 +/− 1.1 |
| Mouse liver +/− (carrier) | 10 | 1.8 +/− 0.86 |
| Pah^{eu2} mouse liver −/− | 6 | 0.0 +/− 0.18 |
| (PKU mouse) | | |

### Example 9

### Ribozyme Directed Against PAH mRNA Lowers the Level of Both mRNA and PAH Enzyme

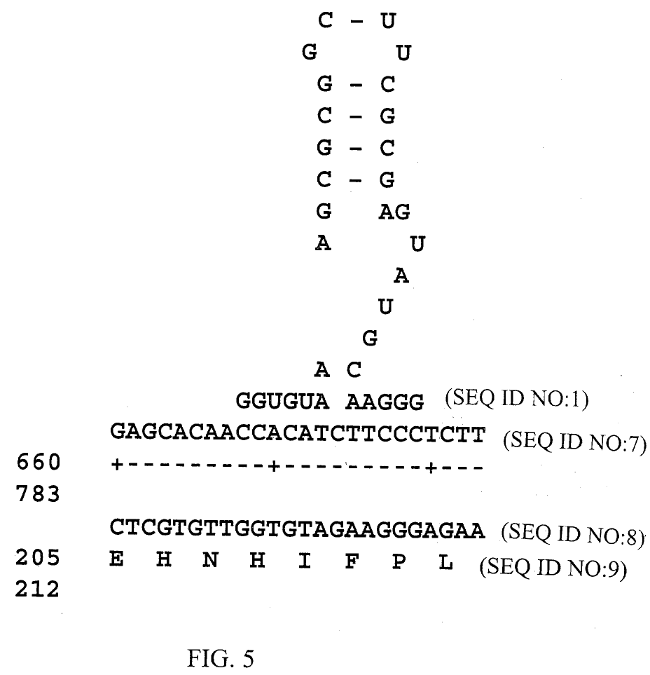

An rAAV-based anti-PAH ribozyme was constructed to reduce endogenous, defective PAH expression. FIG. 5 shows the nucleotide position within the mouse cDNA that is targeted by the ribozyme as well as the ribozyme molecule that binds to the target cDNA. The sequence of this ribozyme molecule is:

5′GGGAACUGAUGAGCGCUUCGGCGCGAAAUGUGG3′ (SEQ ID NO:1). This RNA sequence was changed to DNA and restriction sites were added to each end to put into the rAAV vector (HindIII and SpeI):

| | |
|---|---|
| 5′agcttGGGAACTGATGAGCGCTTCGGCGCGAAATG | (SEQ ID NO:2) |
| TGGa3′ | |
| 3′aCCCTTGACTACTCGCGAAGCCGCGCTTTACACCt | (SEQ ID NO:3) |
| gatc5′ | |

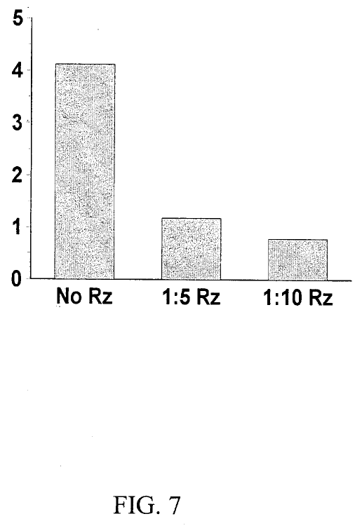

An rAAV vector encoding this ribozyme is shown in FIG. 6. Results from in vitro experiments (FIG. 7) showed that the anti-PAH ribozyme directed against the PAH mRNA lowered PAH mRNAs levels and PAH enzyme activity in the cells.

Mutant ribozymes were designed to check effectiveness, null Ribozyme I (same hybridizing arms) which mutates (*) the active site residue:

| | |
|---|---|
| 5′agcttGGGAACTCATGAGCGCTTCGGCGCGAAATG | (SEQ ID NO:4) |
| TGGa3′ | |
| 3′aCCCTTGATTACTCGCGAAGCCGCGCTTTACACCt | (SEQ ID NO:5) |
| gatc5′ | |

and null Ribozyme 2 (1 change on each arm in addition to the active site change) which is a control for any antisense activity of the ribozyme:

| | |
|---|---|
| 5′agcttGGAAACTCATGAGCGCTTCGGCGCGAAATA | (SEQ ID NO:6) |
| TGGa3′ | |
| 3′aCCTTTGAGTACTCGCGAAGCCGCGCTTTATACCt | (SEQ ID NO:7) |
| gatc5′ | |

The DNA sequence of the PAH gene in the rAAV-CB-mPAH vector was modified so that it produces a functional enzyme, but silent changes in the DNA sequence result in a mRNA that is resistant to the anti-PAH ribozyme. By simultaneous introduction of both the PAH vector and a ribozyme vector, production of functional PAH protein should be enhanced, since the amount of endogenous mutant mRNA and the translated mutant protein subunits will be reduced. Thus, PAH protein produced from the vector-encoded gene will see less negative interference.

### Example 10

### Psuedotyped rAAV-PAH Vectors

Another approach to increasing vector effectiveness is to use either alternate targeting components or alternate subtypes of AAV capsid. A PAH vector packaged into type 5 AAV capsids has been constructed. The pseudotyped vectors have been administered to animals.

### Other Embodiments

It is to be understood that while the invention has been described in conjunction with the detailed description thereof, the foregoing description is intended to illustrate and not limit the scope of the invention, which is defined by the scope of the appended claims. Others aspects, advantages, and modifications are within the scope of the following claims.

What is claimed is:

1. A nucleic acid comprising a phenylalanine hydroxylase-modulating sequence interposed between a first AAV inverted terminal repeat and second AAV inverted terminal repeat, the phenylalanine hydroxylase-modulating sequence being selected from the group consisting of: (a) a polynucleotide encoding a phenylalanine hydroxylase protein and (b) a catalytic polynucleotide that reduces expression of a phenylalanine hydroxylase protein.

2. The nucleic acid of claim 1, wherein the nucleic acid is an expression vector.

3. The nucleic acid of claim 2, wherein the vector is a plasmid.

4. The nucleic acid of claim 1, wherein the nucleic acid is comprised within an rAAV virion.

5. The nucleic acid of claim 1, wherein the phenylalanine hydroxylase-modulating sequence is the polynucleotide encoding a phenylalanine hydroxylase protein.

6. The nucleic acid of claim 5, wherein the phenylalanine hydroxylase protein is capable of catalyzing the intracellular conversion of phenylalanine to tyrosine.

7. The nucleic acid of claim 6, wherein the phenylalanine hydroxylase protein is a wild-type mammalian phenylalanine hydroxylase protein.

8. The nucleic acid of claim 7, wherein the wild-type mammalian phenylalanine hydroxylase protein is a human phenylalanine hydroxylase protein.

9. The nucleic acid of claim 1, wherein the phenylalanine hydroxylase-modulating sequence is the catalytic polynucleotide that reduces expression of a phenylalanine hydroxylase protein.

10. The nucleic acid of claim 9, wherein the catalytic polynucleotide is a ribozyme.

11. The nucleic acid of claim 2, further comprising a promoter operably linked to the phenylalanine hydroxylase-modulating sequence.

12. The nucleic acid of claim 2, further comprising an enhancer element.

13. The nucleic acid of claim 2, further comprising an intron operably linked to the phenylalanine hydroxylase-modulating sequence.

14. The nucleic acid of claim 2, further comprising a woodchuck hepatitis virus post-transcriptional element operably linked to the phenylalanine hydroxylase-modulating sequence.

15. A cell into which the nucleic acid of claim 1 has been introduced.

16. The cell of claim 15, wherein the cell is a mammalian cell.

17. The cell of claim 16, wherein the mammalian cell is a liver cell.

18. A method for modulating phenylalanine hydroxylase activity in a cell, the method comprising the step of administering to the cell an effective amount of the nucleic acid of claim 1.

19. The method of claim 18, wherein the nucleic acid is a vector.

20. The method of claim 19, wherein the vector is a plasmid.

21. The method of claim 20, wherein the nucleic acid is comprised within an rAAV virion.

22. The method of claim 18, wherein the phenylalanine hydroxylase-modulating sequence is the polynucleotide encoding a phenylalanine hydroxylase protein.

23. The method of claim 18, wherein the phenylalanine hydroxylase-modulating sequence is the catalytic polynucleotide that reduces expression of a phenylalanine hydroxylase protein.

24. The method of claim 18, wherein the method comprises introducing into the cell both (a) the polynucleotide encoding a phenylalanine hydroxylase protein and (b) the catalytic polynucleotide that reduces expression of a phenylalanine hydroxylase protein.

25. The method of claim 18, wherein the cell is located in an in vitro culture.

26. The method of claim 18, wherein the cell is within an animal subject.

27. The method of claim 26, wherein the animal subject has a defect in a phenylalanine hydroxylase gene.
